(19) United States
(12) Patent Application Publication
(10) Pub. No.: US 2011/0319794 A1
(43) Pub. Date: Dec. 29, 2011

(54) Convective Energy Transfer into the Eye

(76) Inventor: Michael Gertner, Menlo Park, CA (US)

(21) Appl. No.: 13/007,590

(22) Filed: Jan. 14, 2011

Related U.S. Application Data
(60) Provisional application No. 61/295,685, filed on Jan. 15, 2010.

(51) Int. Cl. A61N 7/00

(52) U.S. Cl. 601/3

(57) ABSTRACT

Methods and devices are described which allow heat to safely be applied to the eyelid of an eye utilizing ultrasound passed through the eyelid. One device preferentially reflects ultrasound at its surface to direct the heat to the inner portion of the eyelid while substantially preventing the heat from contacting the eye. Another device retracts the eyelid from the surface of the eye while applying the heat. Another device applies a cavitating form of ultrasound through the eyelid to enhance opening of the glands within the eyelid. One method further enhances opening of the glands within the eyelid by combining the therapy with bioactive materials on the inside of the gland.

### PRIORITY DATA

This utility patent application claims priority to provisional patent application 61/295,685.

### SUMMARY OF INVENTION

Described below in one embodiment is an ultrasound adjunct for placement inside an eyelid to augment the ability to apply heat to the inside of the eyelid while protecting the surface of the eye.

### Description of Invention

### Incorporation by Reference

Provisional Patent application 61/295,685 is incorporated by reference

### DESCRIPTION OF FIGURES

FIG. 1a depicts and ultrasound probe in proximity to an eyelid.

FIG. 1B depicts an ultrasound adjunct inside an eyelid.

FIG. 1C depicts a tear duct with inspissated material.

FIG. 2A depicts a retractor inside an eyelid

FIG. 2B depicts a retractor with heating elements on it

FIG. 3A-B depicts a method and system to treat eyelids with ultrasound

FIG. 3C depicts ultrasound application to the eye with a contact lens in place.

FIG. 4A depicts another embodiment of a transducer and ultrasound adjunct coupled together.

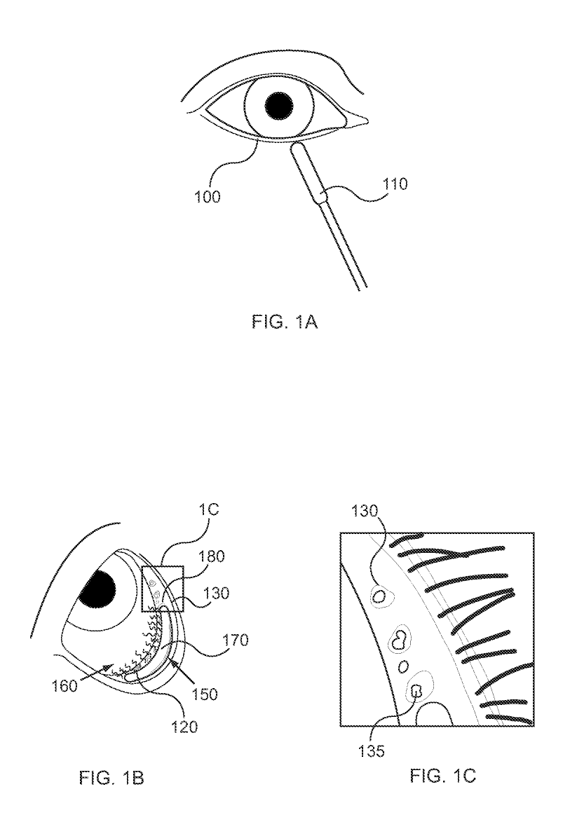

In one embodiment (FIGS. 1A-B), a device 110 is presented in which low frequency ultrasound is applied to the eyelid 100. Ultrasound is applied by the device to the inner eyelid 180 and it travels through the inner eyelid 180. As the ultrasound travels through the eye, in some embodiments, the ultrasonic waves vibrate structures such as tear ducts 130, specifically tear ducts which may be inspissated, or otherwise blocked with material 135 preventing tears from being excreted into the tear film 160 of the eye. In one example, a disease which is treated is dry eye.

FIG. 1B depicts an inner lid 180 and a mebobian gland 130 with material 135 blocking the duct (FIG. 1C). Ultrasound can be used to heat and/or vibrate the material out of the duct 130 (FIG. 1C). In one embodiment, an ultrasound adjunct 170 is utilized to augment the ability of the ultrasound to heat the inner portion of the eyelid and protect the eye. In one embodiment, the adjunct 170 contains an interface which reflects ultrasound heat at the region of the interface between the adjunct and the inner portion of the eyelid 150. Ultrasound is reflected at interfaces and the interface created at the region of the adjunct is transmitted to the inner portion of the eyelid while the eye is protected.

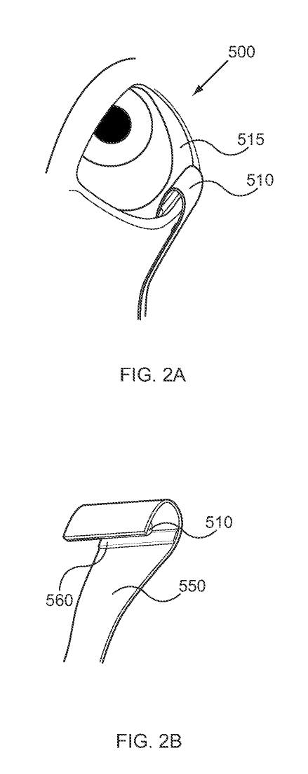

In one embodiment, a method is described in which the lower eyelid is retracted inferiorly by a retractor 510 (FIG. 2A-2B). In this method, the vibration can be delivered through the retracting device 510 and then through the eyelid 515 to the inspissated ducts. In another embodiment, the retractor contains heating or vibratory elements thereon 560; for example, piezoelectric devices 560 can be directly attached to the retractor 550.

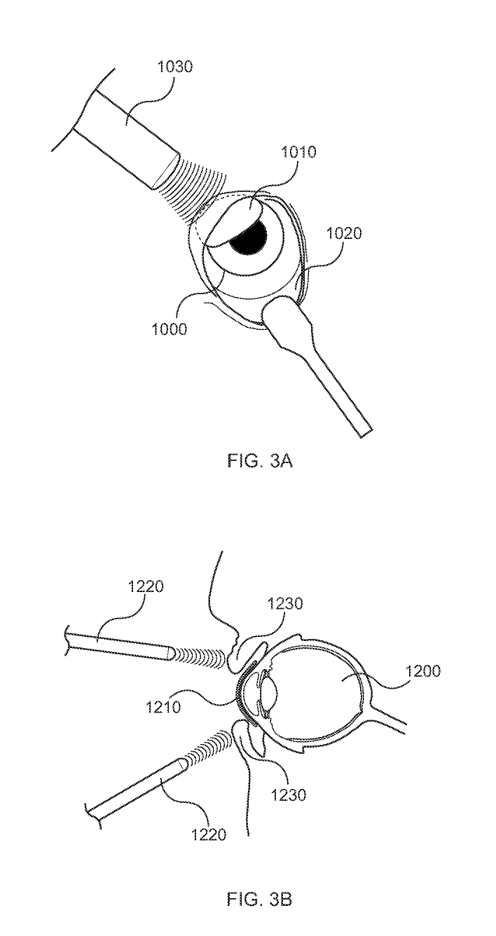

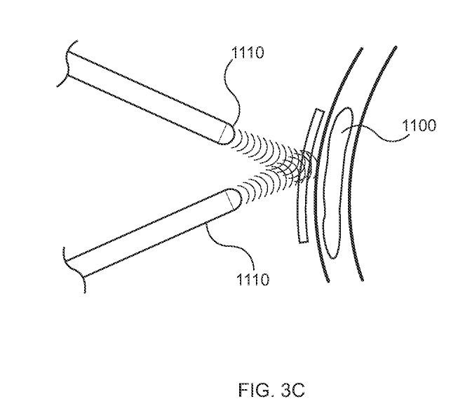

In one embodiment (FIGS. 3A-3B), an insulator 1010 is placed inside the eyelid 1020 or on the eye 1000 to protect or facilitate energy delivery via ultrasound 1030 during the treatment. For example, a device adjunct might be a material that is placed on the inner part of the eyelid or on the lid, the device adjunct 1000 possessing the ability to prevent further propagation of ultrasonic waves from the ultrasonic probe 1030 to enhance delivery of heat to the inner portion of the eyelid and protect the conjunctiva and sclera of the eye from the ultrasound and heat.

In one example, the device adjunct 1010 might consist of an outer portion and an inner portion, the two portions separated by a gas. Ultrasound does not travel through air well and therefore the ultrasound will be reflected from the device adjunct, therefore effectively blocking the ultrasound from reaching the eye yet directing heat to the inner portion of the eyelid.

Furthermore, the interface at the point of internal reflections within the adjunct will begin to heat up as the ultrasonic waves are continually reflected from and within the device adjunct. In this method of treatment, the device adjunct is applied to the inner surface of the eyelid and the ultrasound applied to the outer portion of the eyelid, the waves then transmitting through the eyelid to the mebobian glands to break up the inspissations in the ducts.

In another embodiment (FIG. 3C), a contact lens 1100 is applied to the eye and an ultrasonic energy probe 1110 is applied to the upper and lower eyelids while the eye is closed over the lens 1100. The lens protects the cornea and can be produced such that the ultrasound energy is reflected back to the inner portion of the lid to heat and/or mechanically vibrate the inspissations out of the eyelid.

In another embodiment depicted in FIG. 3B, ultrasound transducer probes 1220 are shown applying ultrasound to eyelids 1230 with a contact lens 1210 underneath the eyelids 1230. The eye 1200 is protected by the contact lens 1210 as well as additional ultrasound reflective layers on the contact lens as described above and below.

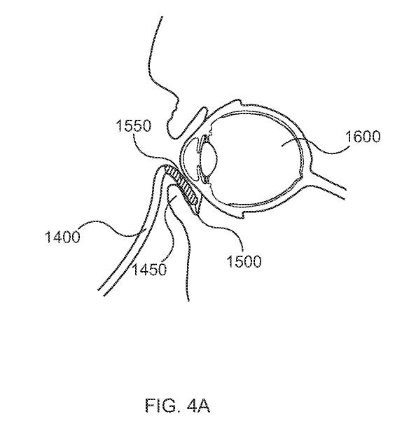

In another embodiment, depicted in FIG. 4A, the ultrasound transducer 1400 and the ultrasound adjunct 1500 are couple around the eyelid 1450. A reflective material or composite of materials 1550 is can be further included in the couple transducer-ultrasound adjunct. As in previous embodiments, the coupled transducer-adjunct surrounds the eyelid 1450 and protects the eye 1600.

In another embodiment, ultrasound is utilized to deliver pharmaceuticals to the eyelids to treat dry eye syndrome. Ultrasound can enhance the delivery of many pharmaceuticals by enhancing their uptake into the cell membranes. In this method, in one embodiment, pharmaceuticals are applied to the inner eyelid of the upper or lower eyelid and subsequently ultrasound is applied to the eyelid to enhance the delivery of the pharmaceutical to the eyelid.

For example, steroids or other anti-inflammatory medications, or other bioactive materials can be applied to the inner lid, to a retractor, or to the ultrasound adjunct placed inside the lid. The bioactive material might further be incorporated into the ultrasound adjunct to, for example, slowly elute from the adjunct to the inner portion of the lid, the ultrasound further augmenting its ability to interact with the glands within the eyelid.

1) A system to apply heat to an eye comprising:
a. An ultrasound reflective material configured to generate heat when ultrasound interacts with the material, the reflective material further configured with a curvature sufficient to be placed on the inner portion of an eyelid and wherein the material further directs heat toward the eyelid and away from the external surface of the eye.

2) The system of claim 1 further comprising an ultrasound probe configured to generate ultrasound waves through the eyelid to interact with the reflective material.

3) The system of claim 2 wherein the ultrasound probe is configured to deliver energy with wavelengths from 100 kHz to 2 MHz.

4) The system of claim 2 wherein the ultrasound probe is physically coupled to the reflective material such that the probe and material can be held on the eyelid.

5) A method of applying heat to the inner portion of an eyelid comprising:
a. Placing an ultrasound adjunct on the inner portion of the eyelid;
b. Applying ultrasound to the outer portion of the eyelid such that the ultrasound will interact with the ultrasound adjunct to generate heat on the inner portion of the eye lid.

6) The method of claim 5 further comprises applying a bioactive agent in combination with the ultrasound adjunct.
